(19) United States
(12) Patent Application Publication
(10) Pub. No.: US 2025/0345010 A1
(43) Pub. Date: Nov. 13, 2025

(54) DUAL DOSE REDUCTION ATTENUATION CORRECTION METHOD AND SYSTEM FOR PET/CT SYSTEM

(71) Applicant: SHENZHEN INSTITUTES OF ADVANCED TECHNOLOGY CHINESE ACADEMY OF SCIENCES, Shenzhen (CN)

(72) Inventors: Zhanli HU, Shenzhen (CN); Zhenxing HUANG, Shenzhen (CN); Wenbo LI, Shenzhen (CN); Dong LIANG, Shenzhen (CN); Hairong ZHENG, Shenzhen (CN); Yongfeng YANG, Shenzhen (CN)

(73) Assignee: SHENZHEN INSTITUTES OF ADVANCED TECHNOLOGY CHINESE ACADEMY OF SCIENCES, Shenzhen (CN)

(21) Appl. No.: 19/278,652

(22) Filed: Jul. 23, 2025

Related U.S. Application Data
(63) Continuation of application No. PCT/CN2023/118838, filed on Sep. 14, 2023.

(51) Int. Cl. A61B 6/03; A61B 6/00

(52) U.S. Cl. CPC A61B 6/037; A61B 6/5235

(57) ABSTRACT

A dual dose reduction attenuation correction method for a PET/CT system includes: performing multi-scale feature extraction on a low-dose ACCT image to obtain a first feature map set

$$\{ {f}_{CT}^{j} \vert j = 1 , 2 , … , N \} ,$$

and performing the multi-scale feature extraction on a low-dose non-attenuation-corrected PET image to obtain a second feature map set

$$\{ {f}_{PET}^{j} \vert j = 1 , 2 , … , N \} ,$$

where N is a set value; performing adaptive spatial alignment between a first feature map

$${f}_{CT}^{j}$$

and a second feature map

$${f}_{PET}^{j} ,$$

and matching and fusing two aligned feature maps to obtain a third feature map set

$$\{ {f}_{CT - PET}^{j} \vert j = 1 , 2 , … , N \} ;$$

performing M iterations of scale-invariant feature extraction on a third feature map

$${f}_{CT - PET}^{N}$$

to obtain an attenuation-corrected feature map

$${f}_{AC - PET}^{N} ,$$

where M is a set value; upscaling a feature map

$${f}_{AC - PET}^{j}$$

to match a size of

$${f}_{CT - PET}^{j - 1} ,$$

and concentrating

$${f}_{CT - PET}^{j - 1}$$

with

$${f}_{AC - PET}^{j}$$

to obtain an attenuation-corrected feature map

$${f}_{AC - PET}^{j - 1} ,$$

where j-N, . . . , 2; and obtaining a visual standard-dose PET image based on a feature map

$${f}_{AC - PET}^{1} .$$

### CROSS-REFERENCE TO RELATED APPLICATIONS

This application is a continuation of International Application No. PCT/CN2023/118838, filed on Sep. 14, 2023, the content of which is incorporated herein by reference in its entirety.

### TECHNICAL FIELD

The present application relates to medical positron emission tomography, and in particular, to a dual dose reduction attenuation correction method and system for a PET/CT system.

### BACKGROUND

Positron Emission Tomography/Computed Tomography (PET/CT) is a widely used medical system in cancer patient management involving diagnosis, monitoring, and follow-up treatment.

In PET imaging, attenuation correction improves visual interpretation and enables accurate quantitative analysis. CT-based methods are commonly used for PET attenuation correction, converting CT Hounsfield units into linear attenuation coefficients at 511 keV through a bilinear relationship; however, these methods have limitations of spatial misalignment between PET and CT images and CT-based artifacts.

Additionally, sequential scanning with PET/CT scanners may expose patients, especially children, to increased radiation risks due to CT dose and potential hazards from overexposure. Although next-generation total-body PET/CT scanners offer unprecedented image quality and quantitative accuracy, with sensitivity improved by approximately 40-fold, they result in higher radiation exposure to patients during total-body scanning compared with single-site examinations.

### SUMMARY

To solve the aforementioned problems in the prior art, the present application aims to provide a dual dose reduction attenuation correction method for a PET/CT system. The method takes into account radiation sources in both PET and CT imaging processes and utilizes a proposed dual dose reduction strategy by reducing both an injection dose and a tube current and using low-dose PET images and low-dose CT images utilized for attenuation correction (ACCT images) to directly generate standard-dose attenuation-corrected (AC) PET images, thereby mitigating radiation risks associated with the PET/CT system. The method is applicable to total-body PET/CT scanners to obtain standard-dose total-body PET images.

To achieve the aforementioned technical objectives, the present application provides the following technical solutions.

In a first aspect, the present application provides a dual dose reduction attenuation correction method for a PET/CT system, including the following steps:

performing multi-scale feature extraction on a low-dose ACCT image to obtain a first feature map set

$$\{ {f}_{CT}^{j} \vert j = 1 , 2 , … , N \} ,$$

and performing the multi-scale feature extraction on a low-dose non-attenuation-corrected (NAC) PET image to obtain a second feature map set

$$\{ {f}_{PET}^{j} \vert j = 1 , 2 , … , N \} ,$$

where N is a set value;
performing adaptive spatial alignment between a first feature map

$${f}_{CT}^{j}$$

and a second feature map

$${f}_{PET}^{j} ,$$

and matching and fusing two aligned feature maps to obtain a third feature map set

$$\{ {f}_{CT - PET}^{j} \vert j = 1 , 2 , … , N \} ;$$

performing M iterations of scale-invariant feature extraction on a third feature map

$${f}_{CT - PET}^{N}$$

to obtain an attenuation-corrected feature map

$${f}_{A C - PET}^{N} ,$$

where M is a set value;

upscaling a feature map

$${f}_{A C - PET}^{j}$$

to match a size of

$${f}_{CT - PET}^{j - 1} ,$$

and concatenating

$${f}_{CT - PET}^{j - 1}$$

with

$${f}_{A C - PET}^{j}$$

to obtain an attenuation-corrected feature map

$${f}_{A C - PET}^{j - 1} ,$$

where j=N, . . . , 2; and
obtaining a visual standard-dose PET image based on a feature map

$${f}_{A C - PET}^{1} .$$

In the above technical solution, an embodiment of the adaptive spatial alignment includes the following steps:
obtaining, based on the first feature map

$${f}_{CT}^{j} ,$$

a first feature map

$${\hat{f}}_{CT}^{j}$$

matching a size of a second feature map

$${f}_{PET}^{j - 1} ,$$

where j=2,3, . . . , N, while for j=1, obtaining a first feature map

$${\hat{f}}_{CT}^{1}$$

matching a size of a cropped low-dose NAC PET image;
performing an affine transformation based on the feature map

$${\hat{f}}_{CT}^{j}$$

to obtain affine transformation parameters (γ, β), where j=1, 2, . . . , N; and
performing channel-wise scaling and shifting operations on the feature map

$${f}_{PET}^{j}$$

using the affine transformation parameters (γ, β) to obtain a second feature map

$${\hat{f}}_{PET}^{j} ,$$

where

$${\hat{f}}_{PET}^{j} = γ {f}_{PET}^{j} + β$$

### and J=1, 2, . . . , N.

In the above technical solution, an embodiment of the step of matching and fusion includes the following steps:
matching the feature maps

$${\hat{f}}_{CT}^{j} and {\hat{f}}_{PET}^{j}$$

through a Hadamard product operation to obtain a feature map

$${\hat{f}}_{CT - PET}^{j} ;$$

obtaining, based on the first feature map

$${f}_{CT}^{j} ,$$

a first feature map

$${{f}^{\hat{ˆ}}}_{CT}^{j}$$

matching the size of the second feature map

$${f}_{PET}^{j - 1} ,$$

where j=2,3, . . . , N, while for j=1, obtaining a first feature map

$${{f}^{\hat{ˆ}}}_{CT}^{1}$$

matching the size of the cropped low-dose NAC PET image; and
fusing the feature maps

$${\hat{f}}_{CT - PET}^{j} and {{f}^{\hat{ˆ}}}_{CT}^{j}$$

through a Hadamard addition operation, where j=1, 2, . . . , N.

In a second aspect, the present application provides a dual dose reduction attenuation correction system for a PET/CT system, where the system employs a dual dose reduction attenuation calibration model that takes a low-dose ACCT image and a low-dose NAC PET image as input, and a standard-dose attenuation-corrected PET image as output;
the dual dose reduction attenuation calibration model includes a first encoder, a second encoder, a spatial alignment module, and a decoder, where
the first encoder is configured to perform multi-scale feature extraction on the low-dose ACCT image to obtain a first feature map set

$$\{ {f}_{CT}^{j} j = 1 , 2 , … , N \} ,$$

where N is a set value;
the second encoder is configured to perform the multi-scale feature extraction on the low-dose NAC PET image to obtain a second feature map set

$$\{ {f}_{PET}^{j} j = 1 , 2 , … , N \} ;$$

the spatial alignment module is configured to perform adaptive spatial alignment between a first feature map

$${f}_{CT}^{j}$$

and a second feature map

$${f}_{PET}^{j} ,$$

and match and fuse two aligned feature maps to obtain a third feature map set

$$\{ {f}_{CT - PET}^{j} \vert j = 1 , 2 , … , N \} ;$$

and
the decoder is configured to upsample a feature map

$${f}_{AC - PET}^{j}$$

to match a size of

$${f}_{CT - PET}^{j - 1} ,$$

and concatenate

$${f}_{CT - PET}^{j - 1} with {f}_{AC - PET}^{j}$$

to obtain an attenuation-corrected feature map

$${f}_{AC - PET}^{j - 1} ,$$

where j=N, . . . , 2; a feature map

$${f}_{AC - PET}^{N}$$

is obtained by performing M iterations of scale-invariant feature extraction on a third feature map

$${f}_{CT - PET}^{N} ;$$

and M is a set value.

In the above technical solution, an embodiment of the first encoder, the second encoder, and the decoder is that they all consist of residual modules, where
each of the residual modules constituting the encoders consists of two three-dimensional convolutional layers, and a batch normalization layer and an activation function layer are disposed between the two convolutional layers; and
each of the residual modules constituting the decoder consists of one three-dimensional transposed deconvolutional layer and one convolutional layer, and the batch normalization layer and the activation function layer are disposed between the three-dimensional transposed deconvolutional layer and the convolutional layer.

In an embodiment of the above technical solution, a loss function for training the dual dose reduction attenuation calibration model is as follows:

$${L}_{MSE} = \frac{1}{n} {\sum }_{i = 1}^{n} {‖ G ( {x}_{i} , {y}_{i} , {θ}_{i} ) - {𝓏}_{i} ‖}_{2}$$

where G represents a mapping relationship; θᵢ represents a network parameter; xᵢ represents a sample from a low-dose ACCT image dataset; yᵢ represents a sample from a low-dose NAC PET image dataset with multiple dose levels; zᵢ represents a sample from a standard-dose attenuation-corrected PET image dataset; and n represents a total number of training samples.

In an embodiment of the above technical solution, the spatial alignment module includes a cropping unit, an affine unit, and a scaling and shifting unit, where
the cropping unit is configured to obtain, based on the first feature map

$${f}_{CT}^{j} ,$$

a first feature map

$${{f}^{\hat{ˆ}}}_{CT}^{j}$$

matching a size of a second feature map

$${f}_{PET}^{j - 1} ,$$

where j=2,3, . . . , N, while for j=1, obtain a first feature map

$${{f}^{\hat{ˆ}}}_{CT}^{1}$$

matching a size of a cropped low-dose NAC PET image;
the affine unit is configured to perform an affine transformation based on a feature map

$${\hat{f}}_{CT}^{j}$$

to obtain attine transformation parameters (γ, β), where j=1, 2, . . . , N; and the scaling and shifting unit is configured to perform channel-wise scaling and shifting operations on the feature map

$${f}_{PET}^{j}$$

using the affine transformation parameters (γ, β) to obtain a second feature map

$${\hat{f}}_{PET}^{j} , where {\hat{f}}_{PET}^{j} = γ {f}_{PET}^{j} + β$$

and j=1, 2, . . . , N.

In an embodiment of the above technical solution, the affine unit is configured to obtain the affine transformation parameters using a filter and a sigmoid function.

In an embodiment of the above technical solution, the spatial alignment module further includes a matching unit and a fusion unit, where
through a the matching unit is configured to match the feature maps

$${\hat{f}}_{CT}^{j} and {\hat{f}}_{PET}^{j}$$

through a Hadamard product operation to obtain a feature map

$${\hat{f}}_{CT - PET}^{j} ;$$

and
through a the fusion unit is configured to fuse the feature maps

$${\hat{f}}_{CT - PET}^{j} and {{f}^{\hat{ˆ}}}_{CT}^{j}$$

through a Hadamard addition operation, where j=1, 2, . . . , N; and the feature map

$${{f}^{\hat{ˆ}}}_{CT}^{j}$$

is re-obtained from the cropping unit.

In an embodiment of the above technical solution, the M iterations of scale-invariant feature extraction are performed using M residual modules on the third feature map

$${f}_{CT - PET}^{N}$$

to obtain the attenuation-corrected feature map

$${f}_{AC - PET}^{N} ,$$

where M is a set value.

The present application has the following beneficial technical effects.

By utilizing a deep learning model to directly generate attenuation-corrected PET images from low-dose NAC PET images and low-dose ACCT images, the present application significantly reduces radiation exposure risks to patients, particularly beneficial for the diagnosis, monitoring, and follow-up treatment of pediatric diseases. When applied to total-body imaging, the present application reduces CT scanning time and can minimize respiratory motion artifacts during CT scanning, thereby ensuring high-quality attenuation-corrected PET images. Therefore, the present application can maintain imaging quality while decreasing harmful radiation doses, demonstrating important scientific significance and promising application prospects in medical diagnostics.

### BRIEF DESCRIPTION OF THE DRAWINGS

To describe the technical solutions in the embodiments of the present application more clearly, the following briefly describes the accompanying drawings required for describing the embodiments. Apparently, the accompanying drawings in the following description show merely some embodiments of the present application, and a person of ordinary skill in the art may still derive other drawings from these accompanying drawings without creative efforts.

FIG. 1 is a schematic diagram of an overall framework of a system in an embodiment.

FIG. 2 is a schematic diagram of image size transformation during spatial alignment in an embodiment.

### DETAILED DESCRIPTION OF THE EMBODIMENTS

PET attenuation correction requires CT images to calculate an attenuation correction coefficient map, resulting in patients being exposed to a relatively high radiation dose. However, most existing methods for addressing low-radiation-dose total-body PET/CT imaging neglect the actual physical characteristics of the device and scanning protocols or fail to consider the issue of radiation dose associated with ACCT.

Therefore, the present application, based on considerations of radiation sources in PET and CT imaging, utilizes a deep learning model to directly generate standard-dose AC PET images from low-dose PET and low-dose ACCT images under the condition of dual-dose reduction while meeting clinical application requirements, thereby mitigating radiation risks associated with a PET/CT system. The technical solutions of the present application can be applied to both partial-body (e.g., brain) and total-body PET attenuation correction without concerns regarding increased radiation exposure. Furthermore, by adopting a dual dose reduction strategy, the CT scanning time is reduced, thereby suppressing respiratory motion artifacts during total-body CT scanning, and further enhancing the quality of PET attenuation correction and improving the quantitative analysis accuracy of PET images.

An explicit and complete description of how to implement the technical solutions of the present application will be given below with reference to the accompanying drawings. Apparently, the described embodiments are only some rather than all of the embodiments of the present application. Based on the embodiments of the present application, all other embodiments obtained by those of ordinary skill in the art without making creative efforts shall fall within the scope of protection of the present application.

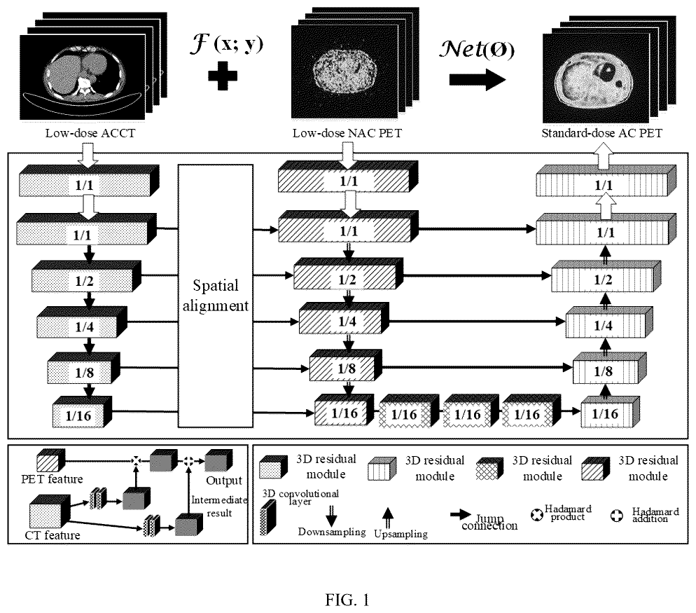

FIG. 1 is a schematic diagram of a dual dose reduction attenuation calibration model employed in the implementation of a dual dose reduction attenuation correction system for a PET/CT system.

The dual dose reduction attenuation calibration model includes a first encoder (first column), a second encoder (second column), a spatial alignment module, and a decoder (third column). As shown in FIG. 1, the first and second encoders perform channel expansion on an input image to increase dimensionality prior to feature extraction. Correspondingly, the decoder finally restores the number of channels to obtain a visual image. For feature map processing, as an example, the first encoder, the second encoder, and the decoder all consist of 5 residual modules.

The first encoder is configured to perform feature extraction on an input low-dose ACCT image, where each of the residual modules is implemented using 2 three-dimensional convolutional layers (a filter size of 3×3×3), with a batch normalization layer and an activation function layer disposed between the two convolutional layers, which can be expressed as follows:

$${f}_{j + 1} = {F}_{j} ( {f}_{j} ) = g ( {w}_{j} {f}_{j} + {b}_{j} )$$

where fⱼ₊ₗ represents a feature map obtained through the residual modules; and Wⱼ and bⱼ represent a weight and bias corresponding to a convolution operation.

The output of the encoder for the low-dose ACCT image can be expressed as follows:

$${f}_{CT} = {F}_{L} ( ⋯ {F}_{1} ( {x}_{i} ) )$$

where f_{CT} represents an output feature map of the ACCT image; xᵢ represents a low-dose ACCT image sample; Fⱼ represents the residual module; and L represents the number of residual modules, thereby obtaining multi-scale features which are collectively denoted by a first feature map set

$$\{ {f}_{CT}^{j} \vert j = 1 , 2 , … , N \} .$$

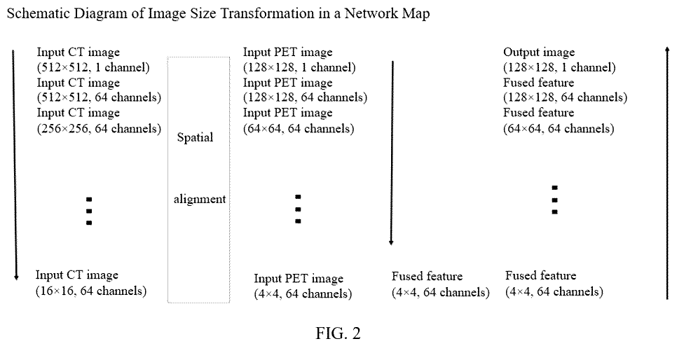

The schematic diagram of image size transformation is shown in the corresponding column of FIG. 2.

Similarly, for a low-dose PET image, the second encoder with the same design is introduced to extract features from an input low-dose NAC PET image, which can be also expressed as follows:

$${f}_{PET} = {F}_{L} ( ⋯ {F}_{1} ( {y}_{i} ) )$$

where f_{CT}=F_{L}( . . . Fₗ(xᵢ)) represents an output feature map of the low-dose NAC PET image; yᵢ represents a low-dose NAC PET image sample; following feature extraction through the residual modules, downsampling is performed through a convolution operation with a kernel size of 3×3×3 and a stride of 2, enabling the network to obtain features at different scales; and the obtained multi-scale features are collectively denoted by

$$\{ {f}_{PET}^{j} \vert j = 1 , 2 , … , N \} .$$

The schematic diagram of image size transformation is shown in the corresponding column of FIG. 2.

The input low-dose ACCT image (512×512) and NAC PET image (192×192) are different in size. Therefore, to address the features of the two modalities, the spatial alignment module is designed to facilitate feature matching and fusion between the low-dose PET and ACCT images across different scales.

First, the NAC PET image is cropped to a processed image size of 128×128. Then, a first feature map

$${f}_{CT}^{j}$$

corresponding to the ACCT image undergoes downsampling twice using a convolutional kernel size of 3×3×3 with a stride of 2, thereby obtaining a feature map

$${\hat{f}}_{CT}^{j}$$

matching a size of

$${f}_{PET}^{j - 1} ,$$

where j=2,3, . . . , N, while for j=1, a first feature map

$${\hat{f}}_{CT}^{1}$$

matching a size of the cropped low-dose NAC PET image is obtained.

Subsequently, based on the feature map

$${\hat{f}}_{CT}^{j} ,$$

an affine transformation is performed to obtain affine transformation parameters (γ, β), where j=1, 2, . . . , N:

$$( γ , β ) = H ( {\hat{f}}_{CT}^{j} )$$

where H represents the affine transformation.

In an embodiment, an ACCT intermediate feature map is processed using a 3×3×3 filter and a sigmoid function to obtain the affine transformation parameters (γ, β), and then the parameters are used to perform channel-wise scaling and shifting operations on a feature map

$${f}_{PET}^{j}$$

of the PET image, which can be specifically expressed as follows:

$${\hat{f}}_{PET}^{j} = γ {f}_{PET}^{j} + β , where j = 1 , 2 , … , N .$$

In the aforementioned spatial alignment module, an adaptive affine transformation is performed along a channel dimension of the intermediate feature map to facilitate feature matching and fusion between the low-dose PET and ACCT images.

The lower left part of FIG. 1 illustrates a matching and fusion method. Specifically, a Hadamard product operation is used to match the feature maps

$${\hat{f}}_{CT}^{j} and {\hat{f}}_{PET}^{j}$$

to obtain a feature map

$${\hat{f}}_{CT - PET}^{j} ;$$

based on the first feature map

$${f}_{CT}^{j} ,$$

a first feature map

$${{f}^{\hat{ˆ}}}_{CT}^{j}$$

matching the size of the second feature map

$${f}_{PET}^{j - 1}$$

is obtained, where j=2,3, . . . , N, while for j=1, a first feature map

$${{f}^{\hat{ˆ}}}_{CT}^{1}$$

matching the size of the cropped low-dose NAC PET image is obtained; and then a map Hadamard addition operation is used to fuse the feature maps

$${\hat{f}}_{CT - PET}^{j} and {{f}^{\hat{ˆ}}}_{CT}^{j} ,$$

where j=1, 2, . . . , N. During this process, when the matching operation is performed between the feature maps

$${f}_{CT}^{j} and {f}_{PET}^{j} ,$$

downsampling is performed twice; following the matching operation, the feature map

$${{f}^{\hat{ˆ}}}_{CT}^{j}$$

is re-obtained from the cropping unit by performing additional downsampling on the feature map

$${f}_{CT}^{j}$$

and then fusion with the matching result to ultimately obtain the output of the spatial alignment module, denoted by a third feature map set

$$\{ {f}_{CT - PET}^{j} \vert j = 1 , 2 , … , N \} .$$

Then, M iterations of scale-invariant feature extraction are performed on a third feature map

$${f}_{CT - PET}^{N}$$

to obtain an attenuation-corrected feature map

$${f}_{AC - PET}^{N} ,$$

where M is a set value. As an example, 3 residual modules connected in series are employed to achieve 3 iterations of scale-invariant feature extraction.

$${f}_{AC - PET}^{N}$$

is input into the decoder, which predicts a standard-dose AC-PET image through corresponding upsampling transposed convolution operations and skip connection operations. The decoder also includes 5 residual modules, each implemented with a three-dimensional transposed deconvolutional layer (using a filter size of 3×3×3 and a stride of 2) and a convolutional layer (using a filter size of 3×3×3 and a stride of 1). Meanwhile, the batch normalization layer and the activation function layer are disposed between the transposed deconvolutional layer and the convolutional layer. The skip connection operations concatenate fused PET and ACCT image features with upsampled features at a corresponding scale, thereby preserving more contextual information.

Referring to the blue column in FIG. 1, from bottom to top, the decoder first upsamples and upscales the feature map

$${f}_{AC - PET}^{N}$$

to match a size of

$${f}_{CT - PET}^{j - 1} ,$$

and concatenates

$${f}_{CT - PET}^{j - 1}$$

with

$${f}_{AC - PET}^{j}$$

through skip connection to obtain an attenuation-corrected feature map

$${f}_{AC - PET}^{j - 1} ,$$

where j=N, . . . , 2. The schematic diagram of image size transformation is shown in the corresponding column of FIG. 2. After obtaining a feature map

$${f}_{AC - PET}^{1} ,$$

channel reduction is performed to obtain a visual standard-dose PET image.

To sum up, the dual dose reduction attenuation correction system for a PET/CT system employs a dual dose reduction attenuation calibration model that takes a low-dose ACCT image and a low-dose NAC PET image as input, and a standard-dose attenuation-corrected PET image as output.

Attenuation correction is a commonly used technique in nuclear medicine imaging (e.g., PET-CT) to correct image brightness variations caused by attenuation of radioactive isotopes. By applying correction to images, it ensures more accurate and reliable radiation absorption across different regions. The present application trained a dual dose reduction attenuation calibration model to enable the correction of low-dose NAC PET images using low-dose ACCT images, thereby obtaining standard-dose attenuation-corrected PET images. The training dataset (including PET and CT data) was from a uEXPLORER total-body PET/CT scanner with a scanning tube voltage of 120 kVp and a tube current of 20 mA. The dataset included total-body PET images at various dose levels without attenuation correction. To simulate noise distribution in the PET images, the noise was classified into four levels, expressed as 1.0%, 2.5%, 5.0%, and 25% of the standard dose. All simulated doses were extracted by randomly selecting a certain proportion of count events from the standard raw data. The reconstructed PET images had a size of 192×192×673 with a slice thickness of 2.9 mm. For the low-dose ACCT images, the scanning tube voltage was 120 k Vp and the tube current was 20 mA, with a slice thickness of 3 mm. The resulting ACCT images had a size of 512×512×673.

A training dataset D={(x₁, y₁), (x₂, y₂), . . . (xₙ, yₙ)} was given, where {x₁, x₂, . . . , xₙ} represents a sample from a low-dose ACCT image dataset; {y₁, y₂, . . . , yₙ} represents a sample from a low-dose NAC PET image dataset; (z₁, z₂, . . . , zₙ) represents a sample from a standard-dose attenuation-corrected PET image dataset; and n represents a total number of training samples.

Then, a loss function for training the dual dose reduction attenuation calibration model was constructed. In an embodiment, a mean squared error (MSE) function was employed as the loss function, where the training loss might be expressed as:

$${L}_{MSE} = \frac{1}{n} {\sum }_{i = 1}^{n} {‖ G ( {x}_{i} , {y}_{i} , {θ}_{i} ) - {𝓏}_{i} ‖}_{2}$$

where G represents a mapping relationship; θᵢ represents a network parameter; xᵢ represents a sample from a low-dose ACCT image dataset; yᵢ represents a sample from a low-dose NAC PET image dataset with multiple dose levels; zᵢ represents a sample from a standard-dose attenuation-corrected PET image dataset; and n represents a total number of training samples.

The dual dose reduction attenuation calibration model was trained using the aforementioned training dataset, with a low-dose NAC total-body PET image yᵢ and a low-dose ACCT image xᵢ as network input, and a standard-dose attenuation-corrected PET image zᵢ as ground truth. An Adam optimizer was used for optimization.

The trained dual dose reduction attenuation calibration model processed the input low-dose ACCT image and low-dose NAC PET image through the following steps:
performing multi-scale feature extraction on the low-dose ACCT image to obtain a first feature map set

$$\{ {f}_{CT}^{j} \vert j = 1 , 2 , … , N \} ,$$

and performing the multi-scale feature extraction on the low-dose NAC PET image to obtain a second feature map set

$$\{ {f}_{PET}^{j} \vert j = 1 , 2 , … , N \} ,$$

where N is a set value;
performing adaptive spatial alignment between a first feature map

$${f}_{CT}^{j}$$

and a second feature map

$${f}_{PET}^{j} ,$$

and matching and fusing two aligned feature maps to obtain a third feature map set

$$\{ {f}_{CT - PET}^{j} \vert j = 1 , 2 , … , N \} ;$$

performing M iterations of scale-invariant feature extraction on a third feature map

$${f}_{CT - PET}^{N}$$

to obtain an attenuation-corrected feature map

$${f}_{AC - PET}^{N} ,$$

where M is a set value;
upscaling a feature map

$${f}_{AC - PET}^{j}$$

to match a size of

$${f}_{CT - PET}^{j - 1} ,$$

and concentrating

$${f}_{CT - PET}^{j - 1}$$

with

$${f}_{AC - PET}^{j}$$

to obtain an attenuation-corrected feature map

$${f}_{AC - PET}^{j - 1} ,$$

where j=N, . . . 2; and
obtaining a visual standard-dose PET image based on a feature map

$${f}_{AC - PET}^{1} .$$

As can be seen from the above implementation process, the dual-dose reduction strategy adopted by the present application reduces radiation doses in total-body PET/CT scanning, holding significant importance for ultra-low-dose scanning protocols in total-body scanning, as it can ensure the integrity of scanning protocols while facilitating greater clinical acceptance by reducing rather than eliminating the ACCT dose for total-body PET attenuation correction. Conventional total-body CT scanning requires approximately 20 seconds, which inevitably introduces respiratory motion artifacts. On the contrary, the dual dose reduction total-body PET attenuation correction strategy disclosed herein can reduce CT scanning time and minimize respiratory motion artifacts during CT scanning, thereby ensuring high-quality attenuation-corrected PET images. The present application can generate clinically acceptable PET images at the minimum dose levels while simultaneously reducing both PET and CT radiation doses.

It should be noted that the technical solutions disclosed herein are applicable to tracer-based PET/SPECT imaging, thereby facilitating the provision of diagnostic references and the formulation of clinical diagnostic guidelines.

In the above-described processes, the terms “first”, “second”, and “third” are intended only for descriptive purposes and should not be construed as indicating or implying relative importance or implying the quantity of technical features indicated. Therefore, a feature limited by “first”, “second”, or “third” may explicitly or implicitly include one or more of such features.

From the description of the above embodiments, a person skilled in the art will clearly understand that the method or system disclosed herein may be implemented by means of software combined with necessary general-purpose hardware, or certainly may be implemented through dedicated hardware including application-specific integrated circuits (ASICs), dedicated CPUs, dedicated memory units, dedicated components, and the like. Generally, any function implemented by computer programs can readily be implemented through corresponding hardware. Moreover, the specific hardware architectures employed to implement the same function may vary, including analog circuits, digital circuits, application-specific circuits, and the like. However, for the present disclosure, software program implementation is preferred in most cases.

It should be noted that reference throughout this specification to “one embodiment”, “another embodiment,” or “an embodiment” means that a particular feature, structure, or characteristic described in connection with the embodiment is included in at least one embodiment of the present application as broadly described herein. Thus, the appearance of the same phrase in various places throughout this specification does not necessarily refer to the same embodiment. Furthermore, when a particular feature, structure, or characteristic is described in connection with any embodiment, it should be understood that any combination with other embodiments to implement such feature, structure, or characteristic shall also fall within the scope of the present application.

Although the foregoing description of the embodiments of the present application has been made with reference to the accompanying drawings, it should be understood that the present application is not limited to the embodiments and application fields described above. The above-described embodiments are merely illustrative and instructive, rather than restrictive. Various other alternative forms may be derived by those of ordinary skill in the art based on the teachings of this specification without departing from the scope of protection defined by the claims of the present application, and these forms shall all fall within the scope of protection of the present application.

What is claimed is:

1. A dual dose reduction attenuation correction method for a Positron Emission Tomography/Computed Tomography (PET/CT) system, comprising the following steps:
performing multi-scale feature extraction on a low-dose ACCT image to obtain a first feature map set
$$\{ {f}_{C T}^{j} \vert j = 1 , 2 , … , N \} ,$$
and performing the multi-scale feature extraction on a low-dose non-attenuation-corrected PET image to obtain a second feature map set
$$\{ {f}_{P E T}^{j} \vert j = 1 , 2 , … , N \} ,$$
wherein N is a set value;
performing adaptive spatial alignment between a first feature map
$${f}_{C T}^{j}$$
and a second feature map
$${f}_{P E T}^{j} ,$$
and matching and fusing two aligned feature maps to obtain a third feature map set
$$\{ {f}_{CT - PET}^{j} \vert j = 1 , 2 , … , N \} ;$$
performing M iterations of scale-invariant feature extraction on a third feature map
$${f}_{CT - PET}^{N}$$
to obtain an attenuation-corrected feature map
$${f}_{AC - PET}^{N} ,$$
wherein M is a set value;
upscaling a feature map
$${f}_{AC - PET}^{j}$$
to match a size of
$${f}_{CT - PET}^{j - 1} ,$$
and concentrating
$${f}_{CT - PET}^{j - 1}$$
with
$${f}_{AC - PET}^{j}$$
to obtain an attenuation-corrected feature map
$${f}_{AC - PET}^{j - 1} ,$$
wherein j=N, . . . , 2; and
obtaining a visual standard-dose PET image based on a feature map
$${f}_{AC - PET}^{1} .$$

2. The method of claim 1, wherein the adaptive spatial alignment comprises the following steps:
obtaining, based on the first feature map
$${f}_{CT}^{j} ,$$
a first feature map
$${\hat{f}}_{CT}^{j}$$
matching a size of a second feature map
$${f}_{PET}^{j - 1} ,$$
wherein j=2,3, . . . , N, while for j=1, obtaining a first feature map
$${\hat{f}}_{CT}^{1}$$
matching a size of a cropped low-dose non-attenuation-corrected PET image;
performing an affine transformation based on the feature map
$${\hat{f}}_{CT}^{j}$$
to obtain affine transformation parameters (γ, β), wherein j=1, 2, . . . , N; and
performing channel-wise scaling and shifting operations on the feature map
$${f}_{PET}^{j}$$
using the affine transformation parameters (γ, β) to obtain a second feature map
$${\hat{f}}_{PET}^{j} ,$$
wherein
$${\hat{f}}_{PET}^{j} = γ {f}_{PET}^{j} + β$$
and j=1, 2, . . . , N.

3. The method of claim 2, wherein the step of matching and fusion comprises the following steps:
matching the feature maps
$${\hat{f}}_{CT}^{j} and {\hat{f}}_{PET}^{j}$$
through a Hadamard product operation to obtain a feature map
$${\hat{f}}_{CT - PET}^{j} ;$$
obtaining, based on the first feature map
$${f}_{CT}^{j} ,$$
a first feature map
$${\hat{\hat{f}}}_{CT}^{j}$$
matching the size of the second feature map
$${f}_{PET}^{j - 1} ,$$
wherein j=2,3, . . . , N, while for j=1, obtaining a first feature map
$${\hat{\hat{f}}}_{CT}^{1}$$
matching the size of the cropped low-dose non-attenuation-corrected PET image; and
fusing the feature maps
$${\hat{f}}_{CT - PET}^{j} and {\hat{\hat{f}}}_{CT}^{j}$$
through a Hadamard addition operation, wherein j=1, 2, . . . , N.

4. A dual dose reduction attenuation correction system for a PET/CT system, wherein
the system employs a dual dose reduction attenuation calibration model that takes a low-dose ACCT image and a low-dose non-attenuation-corrected PET image as input, and a standard-dose attenuation-corrected PET image as output;
the dual dose reduction attenuation calibration model comprises a first encoder, a second encoder, a spatial alignment module, and a decoder,
the first encoder is configured to perform multi-scale feature extraction on the low-dose ACCT image to obtain a first feature map set
$$\{ {f}_{CT}^{j} \vert j = 1 , 2 , … , N \} ,$$
N is a set value;
the second encoder is configured to perform the multi-scale feature extraction on the low-dose non-attenuation-corrected PET image to obtain a second feature map set
$$\{ {f}_{PET}^{j} \vert j = 1 , 2 , … , N \} ;$$
the spatial alignment module is configured to perform adaptive spatial alignment between a first feature map
$${f}_{CT}^{j}$$
and a second feature map
$${f}_{PET}^{j} ,$$
and match and fuse two aligned feature maps to obtain a third feature map set
$$\{ {f}_{CT - PET}^{j} \vert j = 1 , 2 , … , N \} ;$$
and
the decoder is configured to upsample a feature map
$${f}_{AC - PET}^{j}$$
to match a size of
$${f}_{CT - PET}^{j - 1} ,$$
and concatenate
$${f}_{CT - PET}^{j} with {f}_{AC - PET}^{j}$$
to obtain an attenuation-corrected feature map
$${f}_{AC - PET}^{j - 1} ,$$
wherein j=N, . . . ,2; a feature map
$${f}_{AC - PET}^{N}$$
is obtained by performing M iterations of scale-invariant feature extraction on a third feature map
$${f}_{CT - PET}^{N} ;$$
and M is a set value.

5. The system of claim 4, wherein
the first encoder, the second encoder, and the decoder all consist of residual modules,
each of the residual modules constituting the encoders consists of two three-dimensional convolutional layers, and a batch normalization layer and an activation function layer are disposed between the two convolutional layers; and
each of the residual modules constituting the decoder consists of one three-dimensional transposed deconvolutional layer and one convolutional layer, and the batch normalization layer and the activation function layer are disposed between the three-dimensional transposed deconvolutional layer and the convolutional layer.

6. The system of claim 4, wherein
a loss function for training the dual dose reduction attenuation calibration model is as follows:
$${L}_{MSE} = \frac{1}{n} {\sum }_{i = 1}^{n} {‖ G ( {x}_{i} , {y}_{i} , {θ}_{i} ) - {z}_{i} ‖}_{2}$$
G represents a mapping relationship; θᵢ represents a network parameter; xᵢ represents a sample from a low-dose ACCT image dataset; yᵢ represents a sample from a low-dose non-attenuation-corrected PET image dataset with multiple dose levels; zᵢ represents a sample from a standard-dose attenuation-corrected PET image dataset; and n represents a total number of training samples.

7. The system of claim 4, wherein
the spatial alignment module comprises a cropping unit, an affine unit, and a scaling and shifting unit,
the cropping unit is configured to obtain, based on the first feature map
$${f}_{CT}^{j} ,$$
a first feature map
$${\hat{\hat{f}}}_{C T}^{j}$$
matching a size of a second feature map
$${f}_{P E T}^{j - 1} ,$$
j=2,3, . . . , N, while for j=1, obtain a first feature map
$${\hat{\hat{f}}}_{C T}^{1}$$
matching a size of a cropped low-dose non-attenuation-corrected PET image;
the affine unit is configured to perform an affine transformation based on a feature map
$${\hat{f}}_{C T}^{j}$$
to obtain affine transformation parameters (γ, β), j=1, 2, . . . , N; and
the scaling and shifting unit is configured to perform channel-wise scaling and shifting operations on the feature map
$${f}_{PET}^{j}$$
using the attine transformation parameters (γ, β) to obtain a second feature map
$${\hat{f}}_{PET}^{j} , {\hat{f}}_{PET}^{j} = γ {f}_{P E T}^{j} + β$$
and j=1, 2, . . . , N.

8. The system of claim 7, wherein
the affine unit is configured to obtain the affine transformation parameters using a filter and a sigmoid function.

9. The system of claim 7, wherein the spatial alignment module further comprises a matching unit and a fusion unit,
the matching unit is configured to match the feature maps
$${\hat{f}}_{C T}^{j} and {\hat{f}}_{PET}^{j}$$
through a Hadamard product operation to obtain a feature map
$${\hat{f}}_{CT - PET}^{j} ;$$
and
the fusion unit is configured to fuse the feature maps
$${\hat{f}}_{CT - PET}^{j} and {\hat{\hat{f}}}_{CT}^{j}$$
through a Hadamard addition operation, j=1, 2, . . . , N; and the feature map
$${\hat{\hat{f}}}_{C T}^{j}$$
is re-obtained from the cropping unit.

10. The system of claim 4, wherein
the M iterations of scale-invariant feature extraction are performed using M residual modules on the third feature map
$${f}_{CT - PET}^{N}$$
to obtain the attenuation-corrected feature map
$${f}_{AC - PET}^{N} ,$$
M is a set value.
